(19) United States
(12) Patent Application Publication
(10) Pub. No.: US 2008/0248014 A1
(43) Pub. Date: Oct. 9, 2008

(54) Animal Medicament and Method of Manufacture

(75) Inventors: Bruce William Donaldson, Auckland (NZ); Douglas Lang, Tuakau (NL)

Correspondence Address: YOUNG & THOMPSON, 209 Madison Street, Suite 500, ALEXANDRIA, VA, 22314, US

(73) Assignee: Vital Food Processors Limited, Airport Oaks, AUCKLAND (NZ)

(21) Appl. No.: 12/065,281

(22) PCT Filed: Aug. 30, 2006
(86) PCT No.: PCT/NZ06/00220

§ 371 Date: Feb. 29, 2008

(30) Foreign Application Priority Data
Aug. 30, 2005 (NZ) 542100

(51) Int. Cl. A61K 38/43; A61K 47/42

(52) U.S. Cl. 424/94.1; 514/773

(57) ABSTRACT

A medicament for administration to a non-human animal, wherein the medicament includes at Least partially hydrolysed protein and one or more pharmacologically active substances. In a preferred embodiment, the protein is derived from meat which is hydrolysed using a fruit-derived proteolytic enzyme such as actinidin from kiwifruit which enhances the palatability of the resulting hydrolysate. A method of manufacturing the medicament, and a method of medicating a non-human animal by administering to the animal a medicament are also disclosed.

### FIELD OF THE INVENTION

The present invention relates to animal medicaments and methods of manufacture of same, and to methods of medicating non-human animals.

### BACKGROUND OF THE INVENTION

Delivery of pharmaceutically active substances to animals such as companion animals, farm animals, zoo animals or animal pests, in an effective manner is an ongoing problem due to the unpalatability of the substances. Prior art methods for accomplishing such delivery consist of hiding conventional tablets in feed, for example in a piece of meat, or sedating or restraining the animal in order to force the medicament upon it. The prior art also contains methods of producing a meat product with a pocket for insertion of a conventional tablet. The meat product is then fed to the animal in order to administer the medicament.

A major problem with these prior art methods of administering medicaments to animals is that an animal will often reject the meat especially if it has chewed the bait previously and tasted the medicament. There is also a chance that the animal eating the disguised medicament will shy away from completing the meal once the medicament is tasted. This occurrence will result in an animal being given an unknown and/or ineffective dose of the medicament.

### OBJECT OF THE INVENTION

Accordingly, it is an object of the present invention to provide an animal medicament and/or a method of manufacturing an animal medicament and/or a method of medicating a non-human animal which attempts to overcome the problems with the prior art or which will at least provide the public with a useful choice.

### STATEMENT OF THE INVENTION

According to a first aspect of the present invention there is provided a medicament for administration to a non-human animal, wherein the medicament includes at least partially hydrolysed protein and one or more pharmacologically active substances.

The hydrolysed protein may be derived from any protein rich source. For example, the hydrolysed protein may be derived from an animal, bird, fish, shellfish, fungi, insect, plant or bacteria source. Preferably, the hydrolysed protein is derived from meat, fish or biological material.

Examples of suitable sources for the hydrolysed protein include soy, soy meal, milk, whey, shellfish (such as the New Zealand green lipped mussel), chicken skin, meat, fish, and waste products of the meat and fish processing industries.

A particularly suitable source for the hydrolysed protein is mechanically de-boned meat or fish and/or the residue from such a process, which can include, for example, bone, tendons, and fat. This is a low value, inexpensive source of protein which is generally a liability to the meat and fish processing industries as it costs them to dispose of it. Previously it has been uneconomically viable to utilise such waste products so they have generally been discarded at a cost to the industries concerned. In the method of the present invention it is possible to use such waste products as the hydrolysis process enables the proteinaceous material to be fully utilised and form the basis of the medicament.

It is advantageous if the hydrolysed protein is sourced from a food which is appealing to the non-human animal for which the medicament is indicated.

The hydrolysed protein used in the medicament of the present invention may be fully or partially hydrolysed.

Preferably, the protein is hydrolysed using proteolytic enzymes sourced from a plant, animal or bacteria. More preferably, the protein is hydrolysed by plant derived proteolytic enzymes, and even more preferably, fruit derived proteolytic enzymes or fruit derived cysteine proteases. The fruit derived enzyme complexes may be selected from the group consisting of, but not limited to, actinidin, papain, bromelain, and ficin.

The medicament of the present invention may be used to supply any known pharmacologically active substance or substances to a non-human animal. The definition of “pharmacologically active substance” as used herein includes any known pharmaceutical, medicinal, or nutraceutical agent, or any known nutritional or dietary supplement, or any substance or combination of substances which are or are purported to be beneficial to the health or well-being of an animal.

In addition, the term “pharmacologically active substance” includes any known poisons or substance or combination of substances which are or are purported to be adverse to the health or well-being of an animal. For example, it is envisaged that the medicament and methods described herein could be used to control animal pests by administering to the animal pests a medicament as described herein in the form of a poisonous bait or the like. Such a medicament if prepared in accordance with the invention as described herein, would be more readily accepted and consumed by an animal pest than currently known types of poisonous baits and the like. Examples of animal pests which may be treated with the medicament include ferrets, stoats, wild cats, and possums.

In a preferred embodiment the or each pharmacologically active substance may be selected from the group consisting of New Zealand green lipped mussel extract, an enzyme extract, omega-3-oils, an antibiotic, steroids, non-steroidal anti-inflammatory drugs, analgesics, antidepressants, anthelmintics or nutraceuticals.

Examples of pharmacologically active substances which may be used in the present invention include pharmaceuticals such as codeine, cephalosporin, cefadroxil, amoxicillin and the like. An example of a nutritional supplement or nutraceutical is an enzyme extract from kiwifruit, which aids digestion, particularly digestion of protein. A particularly suitable kiwifruit enzyme complex is sold under the brand name ZYACTINASE, and more particularly, ZYACTINASE XRF45, which is manufactured by Vital Food Processors Limited of Auckland, New Zealand.

In a preferred embodiment the or each pharmacologically active substance may be embedded in cyclodextrin molecules. The cyclodextrin molecules may be alpha, beta or gamma cyclodextrin.

In a preferred embodiment the medicament may further include one or more functional polysaccharides. Preferably, the or each functional polysaccharide is selected from the group consisting of fruit polysaccharides, vegetable gums, algal gums, and starches. Examples of functional polysaccharides include guar gum, maize flour and polysaccharides from kiwifruit.

In a preferred embodiment the medicament may further include one or more pharmaceutically acceptable excipients, such as stabilizers, preserving agents (for example, potassium sorbate), colouring agents, sweeteners, flavouring agents and the like.

In a preferred embodiment the medicament may also include one or more fillers or binders. Examples of such fillers or binders include powdered cereal based fillers, rusk, rice, or conventional sausage fillers.

Preferably the medicament is prepared as a dried product, for example, by the use of freeze drying, to form dried biscuits or truffles. A particular advantage of freeze drying the medicament is that it prevents any damage occurring to the pharmacologically active substance or substances contained therein. This is especially true in the situation where the pharmacologically active substance or substances are heat sensitive, for example, in the case of antibiotics, pro-biotics and some enzymes. A further advantage of freeze dried products, is that they do not need to contain preservatives. Some animals, such as cats and dogs, can detect and do not like the taste of preservatives so may not consume products containing preservatives.

Alternatively, the medicament may be prepared in other forms, for example, as a liquid product such as a sauce or gravy.

According to a second aspect of the present invention there is provided a medicament for administration to a non-human animal, wherein the medicament includes at least partially hydrolysed protein and one or more pharmacologically active substances, wherein the hydrolysed protein is derived from meat which has been treated with one or more plant derived proteolytic enzymes.

Preferably the plant derived proteolytic enzymes are fruit derived proteolytic enzymes. Preferably, the fruit derived enzyme complexes are selected from the group consisting of, but not limited to, actinidin, papain, bromelain, and ficin.

Preferably the meat is a waste product of the meat processing industry. For example, a particularly suitable source of meat is mechanically de-boned meat and/or the residue from such a process which is of very little commercial value and would normally be discarded.

Preferably the pharmacologically active substance or at least one of the pharmacologically active substances is an enzyme extract from kiwifruit. More preferably, the kiwifruit enzyme is a ZYACTINASE product, such as ZYACTINASE XRF45.

The pharmacologically active substance or substances may be encapsulated or embedded in cyclodextrin molecules.

Preferably the medicament further includes one or more functional polysaccharides selected from the group consisting of fruit polysaccharides, vegetable gums, algal gums, and starches.

According to a third aspect of the present invention there is provided a medicament for administration to a non-human animal, wherein the medicament includes at least partially hydrolysed protein and one or more pharmacologically active substances, wherein the hydrolysed protein is derived from any protein source which has been treated with proteolytic enzymes derived from kiwifruit, that is, actinidin.

Preferably the hydrolysed protein is derived from an animal, bird, fish, shellfish, fungi, insect, plant or bacteria source.

Examples of suitable sources for the hydrolysed protein include soy, soy meal, milk, whey, shellfish (such as the New Zealand green lipped mussel), chicken skin, meat, fish, and waste products of the meat and fish processing industries.

According to a fourth aspect of the present invention there is provided a method of medicating a non-human animal, wherein the method includes administering to said animal a medicament as described herein.

The non-human animal to be medicated may be selected from the group consisting of carnivorous or omnivorous animals. For example, a carnivorous animal may be a dog, a cat, a snake, or a marine mammal. An example of an omnivorous non-human animal for treatment with the medicament according to the present invention is a chimpanzee, a bear, a pig, or a rat.

The medicament could be used to control animal pests by administering to the animal pests a medicament as described herein in the form of a poisonous bait or the like. Such a medicament if prepared in accordance with the invention as described herein would be more readily accepted and consumed by an animal pest than currently known types of poisonous baits and the like. Examples of animal pests which may be treated with the medicament include ferrets, stoats, wild cats, and possums.

According to a fifth aspect of the present invention there is provided a method of manufacturing a medicament for administration to a non-human animal, said method including the steps of:

treating a protein source with one or more proteolytic enzymes to produce at least partially hydrolysed protein;
pasteurizing said hydrolysed protein;
mixing or blending a selected amount of one or more pharmacologically active substances into said hydrolysed protein;
forming the hydrolysed protein into a paste;
extruding or allocating the paste into selected dosage sizes;
drying said paste to form the resulting medicament.

Preferably the protein source is derived from waste meat from the meat processing industry, such as mechanically de-boned meat and/or the residue from such a process.

Preferably the or each proteolytic enzyme is a plant derived proteolytic enzyme, and more preferably a fruit derived proteolytic enzyme or enzyme complex. Preferably the fruit derived enzyme complex is selected from the group consisting of, but not limited to, actinidin, papain, bromelain, and ficin.

Preferably the paste is freeze dried to form the resulting medicament. A particular advantage of freeze drying the medicament is that it prevents any damage occurring to the pharmacologically active substance or substances contained therein. This is especially true in the situation where the pharmacologically active substance or substances are heat sensitive, for example, in the case of antibiotics, pro-biotics and some enzymes.

Alternatively, the paste may be dried by other drying processes known in the art, for example, spray drying.

The present invention therefore provides a pre-prepared drug delivery system or medicament for animals which is palatable or “treat-like”. By hydrolysing protein, particularly uneconomically viable, low value waste meat protein, with a plant derived enzyme complex such as actinidin from kiwifruit, it is possible to produce a cost-effective, organic hydrolysed product which contains one or more pharmacologically active substances and which is digestible, nutritional and palatable to animals and does not have the bitter taste which is associated with other forms of hydrolysed protein.

Further aspects and advantages of the present invention will become apparent from the ensuing description which is given by way of example only.

### DETAILED DESCRIPTION

In accordance with the present invention there is provided a medicament for administration to a non-human animal, wherein the medicament includes at least partially hydrolysed protein and one or more pharmacologically active substances. The present invention also provides a method of medicating a non-human animal, wherein the method includes administering to said animal a medicament as described herein.

The hydrolysed protein useable in the invention may be derived from any protein rich source. For example, the hydrolysed protein may be derived from an animal, bird, fish, shellfish, fungi, insect, plant or bacteria source. Preferably, the hydrolysed protein is derived from meat, fish or biological material. Examples of suitable sources for the hydrolysed protein include soy, soy meal, milk, whey, shellfish (such as the New Zealand green lipped mussel), chicken skin, meat, fish, and waste products of the meat and fish processing industries.

A particularly suitable source for the hydrolysed protein is mechanically de-boned meat or fish and/or the residue from such a process which can include, for example, bone, tendons, and fat. This provides a hydrolysed protein of high nutritional value as not only the flesh/meat components may be used, but also the bones, bone marrow, connective tissue and cartilaginous material.

It is advantageous if the hydrolysed protein is sourced from a food which is appealing to the non-human animal for which the medicament is indicated. If the hydrolysed protein for use in the medicament is sourced from a food which is appealing to the animal then the hydrolysed protein will also contain flavour volatile compounds which will appeal to the animal to be medicated. For example, if the animal to be medicated is a cat, a possible source of hydrolysed protein for use in the medicament may be fish or chicken. Different circumstances may result in the availability of different protein sources for hydrolysis. If the protein to be hydrolysed is of a type which is normally eaten by the non-human animal to be medicated and hydrolysis is partial then a more suitable and palatable product may be obtained.

The hydrolysed protein used in the medicament of the present invention may be fully or partially hydrolysed.

The hydrolysed protein may be hydrolysed by any means known to one skilled in the art. However, preferably the protein is hydrolysed by proteolytic enzymes. These proteolytic enzymes may be sourced from a plant, bacteria, or animal. In preferred embodiments of the present invention the protein hydrolysis is carried out by plant derived proteolytic enzymes. This use of plant derived enzymes avoids any likelihood of contamination from for example other meat sources (BSE). The use of plant derived enzymes also avoids legislation and public opinion restricting or directed against the use of enzymes derived from farmed animals or modified micro-organisms. More preferably, the protein is hydrolysed by fruit derived proteolytic enzymes, or fruit-derived cysteine proteases. The fruit derived enzyme complexes may be selected from the group consisting of, but not limited to, actinidin, papain, bromelain, and ficin.

Most preferably, the enzymes used are obtained from the fruit of Actinidia (or kiwifruit) species, such as Actinidia chinensis or Actinidia deliciosa. A particularly suitable enzyme complex for use in this invention is that manufactured by Vital Food Processors Limited of Auckland, New Zealand, under the brand name PROACTINASE, which contains actinidin.

The protein is hydrolysed by exposing the protein source to one or more of the proteolytic enzymes such that a protein rich extract in the form of a liquid, such as a broth is produced, and then deactivating the enzymes by subjecting the broth to high temperatures, for example, greater than 60 degrees Celsius, for at least five minutes. As well as providing for the deactivation of the enzymes, the heating step facilitates solubilisation of the protein and the separation of any fats and oils. The fatty or oily layers may be siphoned off. Mechanical agitation of the mixture prior to or during the heating step may be utilised. This may aid in separating proteinaceous material from bones and the like and may facilitate the liquefaction of the proteins. Preferably, the resulting hydrolysate is in the form of a smooth paste, or is formed into a paste by the addition of suitable excipients, such that it is malleable and able to be extruded.

The resulting hydrolysate is a sterile, pasteurized liquid having reduced pathogen loading due to destruction of pathogens by both heat and hydrolysis. Added to this advantage is that the hydrolysate does not have the bitter taste caused by bitter peptides which is attributed to most other forms of hydrolysed protein. This is a result of the particular methodology used in that the enzyme complex when applied to the protein source produces a hydrolysate which is devoid of the peptides that impart the classic bitter aftertaste associated with most commercial protease reacted hydrolysates. This increased palatability factor is a significant advantage.

Other advantages associated with this method of hydrolysis is that it allows for cost effective recovery of the proteinaceous material from the protein source. For example, if mechanically de-boned meat residue is used as the protein source, it is inexpensive as it is a waste product produced by commercial mechanical de-boning machines, and therefore does not command a price premium like meat does. By using the residue from such a process, for example, the boned out carcass frame (which can be put through a crusher) and/or the bone mash it is possible to hydrolyse it to produce an organic product which some pet owners are willing to pay a premium for. This enables the recovery of high value organic components from a low value meat/protein source which is otherwise not commercially viable and is usually discarded.

The recovered proteinaceous liquid meat from this source is therefore of low cost and eminently suitable for use in medicated “truffles” or biscuits where pet owners are prepared to pay a premium for products of organic origin, especially if the medicament is one they expect to feed to their pets on an ongoing basis as part of a daily diet, such as glucosamine for joint problems, omega3 for inflammatory problems or protease enzymes for digestive problems.

The liquid meat can also be produced without the influence of genetic modification, and is therefore 100% natural and able to be consumed daily by cherished pets, valuable working dogs, guard dogs, guide dogs and zoo animals and the like without fear of possible longer term detrimental health repercussions or side effects.

The resulting hydrolysate is suitable for moisture adaptation by spray-drying, freeze-drying or other technology, and therefore can be prepared into the required dosage form, such as, for example, dried biscuits or “truffles”. Preferably the medicament is prepared as a dried product, and more preferably a freeze dried product. A particular advantage of freeze drying the medicament is that it prevents any damage occurring to the pharmacologically active substance or substances contained therein. This is especially true in the situation where the pharmacologically active substance or substances are heat sensitive, for example, in the base of antibiotics, pro-biotics and some enzymes. A further advantage of freeze dried products is that they do not need to contain preservatives. Some animals, such as cats and dogs, can detect and do not like the taste of preservatives so may not consume products containing preservatives.

Alternatively, the medicament may be prepared in other forms, for example, as a liquid product such as a sauce or gravy.

The medicament of the present invention may be used to supply any known pharmacologically active substance to a non-human animal. In a preferred embodiment the pharmacologically active substance or substances may be selected from the group consisting of New Zealand green lipped mussel extract, an enzyme extract, omega-3-oils, an antibiotic, steroids, non-steroidal anti-inflammatory drugs, analgesics, antidepressants, anthelmintics or nutraceuticals.

Examples of pharmacologically active substances which may be used in the present invention include pharmaceuticals such as codeine, cephalosporin, cefadroxil, amoxicillin and the like. An example of a nutritional supplement or nutraceutical is an enzyme extract from kiwifruit, which aids digestion, particularly digestion of protein. A particularly suitable kiwifruit enzyme complex is sold under the brand name ZYACTINASE, and more particularly, ZYACTINASE XRF45, which is manufactured by Vital Food Processors Limited of Auckland, New Zealand.

The method of the present invention is particularly advantageous where the non-human animal would not choose to consume the pharmacologically active substance or substances. For especially unpleasant (for example, bitter tasting) pharmacologically active substances, extra steps can be taken to reduce the nature of such flavour. For example, in order to reduce the unpleasantness of a flavour the agent may be encapsulated or embedded in another molecule, for example, a cyclodextrin molecule, such as alpha, beta or gamma cyclodextrin.

The medicament may further include one or more functional polysaccharides. The one or more functional polysaccharides may be used in the manufacture of the medicament in order to provide binding stability to the resultant medicament. The or each functional polysaccharide may be selected from the group consisting of fruit polysaccharides, vegetable gums, algal gums, and starches. Examples of functional polysaccharides include guar gum, maize flour, and polysaccharides from kiwifruit.

The medicament may also further include one or more pharmaceutically acceptable excipients, such as stabilizers, preserving agents (for example, potassium sorbate), colouring agents, sweeteners, flavouring agents and the like.

The medicament may also include one or more fillers or binders. Examples of such fillers or binders include powdered cereal based fillers, rusk, rice, or conventional sausage fillers.

The present invention also provides a method of manufacturing a medicament for administration to a non-human animal. The method generally includes the steps of treating a protein source with one or more proteolytic enzymes to produce at least partially hydrolysed protein in liquid form. The hydrolysed protein is then pasteurized, preferably by subjecting it to high temperatures, for example, by passing it through a heat exchanger. It is then cooled and mixed or blended with a selected amount of one or more pharmacologically active substances. At this stage other excipients such as fillers, binders, preserving agents, stabilizers etc may be added to the hydrolysed protein. The hydrolysed protein is formed into a paste, either as a result of the hydrolysis process, or by adding suitable excipients to form a paste. The paste is then extruded or allocated into selected dosage sizes and then dried to form the resulting medicament.

Preferably the protein source is derived from waste meat from the meat processing industry, such as mechanically de-boned meat and/or the residue from such a process.

Preferably the or each proteolytic enzyme is a plant derived proteolytic enzyme and more preferably, a fruit derived enzyme complex such as the kiwifruit enzyme, actinidin.

Preferably the paste is freeze dried to form the resulting medicament. A particular advantage of freeze drying the medicament is that it prevents any damage occurring to the pharmacologically active substance or substances contained therein. This is especially true in the situation where the pharmacologically active substance or substances are heat sensitive, for example, in the case of antibiotics, pro-biotics and some enzymes.

Alternatively, the paste may be dried by other drying processes known in the art, for example, spray drying.

Some examples of typical formulations of the invention will now be described. It must be understood that these are examples only and are in no way intended to limit the spirit and scope of the invention.

### EXAMPLE 1

A protein rich flowable hydrolysate is made by exposing a protein source such as beef, lamb, chicken, or venison to plant derived cysteine proteases. The hydrolysed protein is then pasteurised by passing through a heat exchanger and is cooled and combined with maize flour and guar gum to form a paste. The paste is then blended with potassium sorbate, and with the appropriate amount of the pharmacologically active substance or substances according to the desired final formulation.

The cooled medicated paste is then extruded into trays, frozen, and then freeze dried. The final freeze dried product is packed into foil bags, is nitrogen flushed, and sealed under a light vacuum in order to prevent spoilage.

### EXAMPLE

### K-9 Kisses

| Amount | Ingredient |
|---|---|
| 11 kg | Hydrolysed protein |
| 7 kg | Cereal/Binder |
| 200 gm | Omega-3-oil |
| 120 gm | ZYACTINASE |

The ingredients listed above are mixed according to the methodology previously discussed. The average weight of an individual dose for a 7 kg to 15 kg dog is 6.5 g. The dry weight of this dose following lyophilisation (freeze drying) is approximately 3.5 g. Such a dose can be easily handled by the pet owner and may be sold in packs suitable, for example, for a week or a month's worth of medication.

### EXAMPLE 2

A protein rich flowable hydrolysate is made by exposing a protein source such as beef, lamb, chicken, or venison to a fruit derived protease enzyme. The hydrolysed protein is pasteurized by passing through a heat exchanger then rapidly cooled to produce a sterile, chilled, liquid meat. This can be further stabilized for longer term shelf stability by the addition of a preserving agent such as but not limited to potassium sorbate.

The chilled liquid meat is then blended with a mixture containing, but not limited to, the following ingredients:

a cereal based filler such as that used in conventional sausage manufacture;
a further binding agent such as a food type gum, for example, guar gum;
an appropriate amount of medication.

In general the liquid meat and binder is mixed in the ratio of 12 parts of liquid meat to 7 parts binder to form the medication carrier.

The medication is added to the carrier at a dose appropriate rate.

For example, if a fruit protease complex such as ZYACTINASE XRF45 was to be incorporated in the carrier base to produce medicated “truffles” to improve protein digestion in domestic dogs, the weight of the dogs being treated would be defined and the amount of medication required for each treatment would be calculated.

For example, a typical formulation used for the treatment of dogs in the weight range of 7 kg to 15 kg may be as follows:

| Amount | Ingredient |
|---|---|
| 12 kg | Protein hydrolysate consisting of |
| | approx 70% meat, 20% fat and 10% |
| | added water |
| 7 kg | Cereal based binder |
| 0.25 kg | ZYACTINASE XRF45 powder |

This would make approximately 2,950 “truffles” each of 6.5 g (wet weight). Each truffle would contain approximately 85 mg of the pharmacologically active substance.

### EXAMPLE 3

The following example formulation contains codeine as the pharmacologically active substance.

| Amount | Ingredient |
|---|---|
| 12 kg | Protein hydrolysate consisting of |
| | approx 70% meat, 20% fat and 10% |
| | added water |
| 7 kg | Cereal based binder |
| 0.06 kg | Codeine |

This would make approximately 2,950 “truffles” each of 6.5 g (wet weight). Each truffle would contain approximately 20 mg of the pharmacologically active substance.

### EXAMPLE 5

The following example formulation contains an orally administered Cephalosporin such as Cefadroxil (which is claimed to be safer than the more common antibiotics like Amoxicillin) as the pharmacologically active substance.

| Amount | Ingredient |
|---|---|
| 12 kg | Protein hydrolysate consisting of |
| | approx 70% meat, 20% fat and 10% |
| | added water |
| 7 kg | Cereal based binder |
| 0.150 kg | Cefadroxil |

This would make approximately 2,950 “truffles” each of 6.5 g (wet weight). Each truffle would contain approximately 50 mg of the pharmacologically active substance, and a 10 kg dog would require one truffle four times per day.

### Advantages

Thus it can be seen that a medicament has been provided which is able to be easily administered to a non-human animal. Due to the palatability of the hydrolysed protein used in the medicament of the present invention, the animal will not reject the medicament, nor will it shy away from completing the food. Rather, the animal will readily accept the medicament. Accordingly, the animal will always be given a known and effective dose of the medicament. It is a simple matter to give a dog a truffle or biscuit every day or every few hours as necessary, but it is quite a different matter to have to inject the dog or restrain it and force it to consume a tablet or the like.

In addition, the medicament is cost-effective to produce, particularly if waste products from meat or fish processing industries are used as the protein source, as this is a low value, inexpensive source of protein which is otherwise not commercially viable and would be discarded. Instead, these waste products can be used in the present invention as the hydrolysis process enables the proteinaceous material to be fully utilised and form the basis of the medicament. In addition, the medicament is 100% natural, organic, and is of high nutritional value.

A further advantage is realised if the medicament is freeze dried in that freeze drying prevents any damage occurring to the pharmacologically active substance or substances contained therein, and also freeze dried products do not need to contain preservatives.

The present invention therefore provides a pre-prepared drug delivery system or medicament for animals which is palatable or “treat-like”. By hydrolysing protein, particularly uneconomically viable, low value waste meat protein, with a plant derived enzyme complex such as actinidin from kiwifruit, it is possible to produce a cost-effective, organic hydrolysed product which contains one or more pharmacologically active substances and which is digestible, nutritional and palatable to animals and does not have the bitter taste which is associated with other forms of hydrolysed protein.

### VARIATIONS

Aspects of the present invention have been described by way of example only and it should be appreciated that modifications and additions may be made thereto without departing from the scope thereof.

It is to be understood that the scope of the invention is not limited to the described embodiments and therefore that numerous variations and modifications may be made to these embodiments without departing from the scope of the invention as set out in the specification. Wherein the foregoing description reference has been made to integers or components having known equivalents then such equivalents are herein incorporated as if individually set forth.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

1. A medicament for administration to a non-human animal, wherein the medicament includes at least partially hydrolysed protein and one or more pharmacologically active substances.

2. A medicament as claimed in claim 1, wherein the hydrolysed protein is derived from meat which has been treated with one or more plant derived proteolytic enzymes.

3. A medicament as claimed in claim 2, wherein the plant derived proteolytic enzymes are fruit derived proteolytic enzymes.

4. A medicament as claimed in claim 3, wherein the fruit derived proteolytic enzymes are selected from the group consisting of actinidin, papain, bromelain, and ficin.

5. A medicament as claimed in claim 2, wherein the meat is a waste product from the meat processing industry, such as mechanically de-boned meat and/or the residue from such a process.

6. A medicament as claimed in claim 1, wherein the Pharmacologically active substance or at least one of the pharmacologically active substances is an enzyme extract from kiwifruit.

7. A medicament as claimed in claim 6, wherein the kiwifruit enzyme extract is a ZYACTINASE product, such as ZYACTINASE XRF45.

8. A medicament as claimed in claim 1, wherein the pharmacologically active substance or substances are encapsulated or embedded in cyclodextrin molecules.

9. A medicament as claimed in claim 1, wherein the medicament further includes one or more functional polysaccharides selected from the group consisting of fruit polysaccharides, vegetable gums, algal gums, and starches.

10. A method of medicating a non-human animal, wherein the method includes administering to said animal a medicament as claimed in claim 1.

11. A method of manufacturing a medicament for administration to a non-human animal, said method including the steps of:
treating a protein source with one or more proteolytic enzymes to produce at least partially hydrolysed protein;
pasteurizing said hydrolysed protein;
mixing or blending a selected amount of one or more pharmacologically active substances into said hydrolysed protein;
forming the hydrolysed protein into a paste;
extruding or allocating the paste into selected dosage sizes;
drying said paste to form the resulting medicament.

12. A method of manufacturing a medicament for administration to a non-human animal as claimed in claim 11, wherein the or each proteolytic enzyme is selected from the group consisting of plant derived proteolytic enzymes, including fruit derived enzyme complexes.

13. A method of manufacturing a medicament for administration to a non-human animal as claimed in claim 11, wherein the paste is freeze dried to form the resulting medicament.

14. A method of manufacturing a medicament for administration to a non-human animal as claimed in claim 12, wherein the paste is freeze dried to form the resulting medicament.
